(19) United States
(12) Patent Application Publication
(10) Pub. No.: US 2012/0174931 A1
(43) Pub. Date: Jul. 12, 2012

(54) PHARYNX PROTECTOR

(76) Inventors: Andreas Nilsson, Lund (SE); Jan-Ove Persson, Hoor (SE)

(21) Appl. No.: 13/496,884

(22) PCT Filed: Sep. 15, 2010
(86) PCT No.: PCT/EP10/63519

§ 371 Date: Mar. 18, 2012

(30) Foreign Application Priority Data
Sep. 18, 2009 (SE) 0950683-3

(51) Int. Cl. A61B 19/00

(52) U.S. Cl. 128/846

(57) ABSTRACT

A pharynx protector may include a proximal end and a distal end, the distal end being adapted to be inserted into an oesophagus of a patient. The protector may include an elongated tubular part and a distally located receiving portion for receiving a puncture needle during a laryngectomy, wherein the tubular part is substantially flexible. The receiving portion may include a first opening, communicating with a lumen of the tubular part, such that a guide wire may be inserted through the first opening and further passed proximally out of the pharynx protector. A surface of an inner wall may be of a rigid material.

### CROSS-REFERENCE TO RELATED APPLICATIONS

This application claims priority to Sweden Patent Application 0950683-3 filed on Sep. 18, 2009 and PCT/EP2010/063519 filed on Sep. 15, 2010, which are hereby incorporated by reference in their entireties.

### TECHNICAL FIELD

This invention pertains in general to the field of a pharynx protector, for protecting the oesophagus and/or pharynx during performance of a secondary puncture in a laryngectomized patient, said pharynx protector having a proximal end and a distal end, said distal end being adapted to be inserted into the oesophagus of a patient, said pharynx protector comprising a proximally located elongated tubular part and a distally located receiving portion.

### BACKGROUND

In the field of laryngectomy a voice prosthesis is often used for tracheoesophagal speech. The voice prosthesis is then placed in a puncture in the tracheoesophageal wall. The voice prosthesis may be placed in said puncture when the voice box is removed and the trachea is sutured to the skin of the neck—so called primary puncture—or after time of surgery, under general anesthesia—so called secondary puncture. A voice prosthesis has a tubular body, with a flange in each end. The tubular body is to be mounted in the tracheoesophageal wall with a flange situated on the tracheal side, substantially hindering movement of the voice prosthesis into the oesophagus, and the other flange situated on the oesophagal side, hindering movement of the voice prosthesis into the trachea. A valve member is located in the lumen of the tubular body. The voice prosthesis may also be provided with a safety strap, arranged on the flange intended to be situated on the tracheal side.

Laryngectomy may for example be performed in cases of laryngeal cancer.

To create a tracheoesophageal puncture during primary puncture, a pharynx protector is inserted caudal in the pharynx until the tip of the pharynx protector reaches the intended puncture site in the esophagus. The pharynx protector is a hollow, rigid, and cylindrical device with a handle. It is inserted in the pharynx/esophagus to protect the posterior wall during puncture. The tip of the device has normally an oblique opening, which is palpated by the surgeon to verify the correct position for puncture. The device has a slot running from the oblique opening along the top of the cylindrical part to enable radial puncturing for surgeons who prefer doing so. The tip of the pharynx protector is palpated through the trachea to verify the correct placement of the puncture. The puncture is made with a puncture needle through the tracheoesophageal wall against the pharynx protector. The puncture needle is a thick and hollow needle normally made of steel. The needle is used to create the puncture and to facilitate the subsequent introduction of a guide wire, which is inserted through the puncture needle.

The guide wire is normally a plastic thread, which is plastically deformable. The puncture needle may have a bent tip in order to direct the guide wire into the hollow cylindrical part of the pharynx protector. The puncture needle is oriented so that a bent tip thereof directs a guide wire—subsequently inserted through the puncture needle—into the lumen of the pharynx protector. Next, the guide wire is introduced through the puncture needle until the distal tip of the guide wire extends approximately 20 cm through the pharynx protector. The puncture needle and the pharynx protector are removed, leaving the guide wire in place through the puncture of the tracheoesophageal wall. Thereafter, a voice prosthesis is arranged on the guidewire and pulled through the tracheoesophageal puncture.

During secondary puncture a rigid esophagoscope is generally inserted in the esophagus instead of a pharynx protector until the tip of the esophagoscope can be palpated at the puncture site. The puncture is then made with the puncture needle against the esophagoscope which acts as a pharynx protector.

U.S. Pat. No. 6,159,243 discloses a voice prosthesis implantation kit for secondary puncture including; a pharynx protector in form of a leader element, which can be introduced via the mouth to the location where the voice prosthesis is to be implanted. The leader element comprises an expansion element in form of a balloon, which balloon shall interact with a cutting element carrying a dilator for a voice prosthesis. The kit according to U.S. Pat. No. 6,159,243 is only usable for secondary puncture. However, the pharynx protector according to U.S. Pat. No. 6,159,243 is accompanied by several drawbacks. For example, this pharynx protector comprises several parts, and a complicated configuration of these parts, such as the need of a pressure applying device for expanding the balloon; the positioning of the cutting device is unguided, and has to be exactly positioned within the balloon, not to risk that the back wall of the oesophagus from being penetrated.

Hence, an improved pharynx protector would be advantageous, and in particular a pharynx protector allowing for use in secondary puncture; said pharynx protector being easy to manufacture and assemble, leading to a lower production cost, providing a safe way to penetrate the front wall of the oesophagus without risking injuring other parts of the oesophagus, and providing a pharynx protector that is more pleasant for the patient during insertion into the oesophagus, would be advantageous.

### SUMMARY

Accordingly, the present invention preferably seeks to mitigate, alleviate or eliminate one or more of the above-identified deficiencies in the art and disadvantages singly or in any combination and solves at least the above mentioned problems by providing a pharynx protector for protecting the oesophagus and/or pharynx during performance of a laryngectomy, said pharynx protector having a proximal end and a distal end, said distal end being adapted to be inserted into the oesophagus of a patient, said pharynx protector comprising an elongated tubular part and a distally located receiving portion, wherein said tubular part being flexible, and said receiving portion comprising; an opening, communicating with the lumen of the tubular part, such that a guide wire may be inserted through the opening and further passed proximally out of the pharynx protector; and a surface of the inner wall, having a normal passing through said opening, being of a rigid material.

Advantageous features hereof are embodied in the dependent claims.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIGS. 1a to 1d illustrate a pharynx protector according to one embodiment of the present invention;

FIGS. 2a to 2c illustrate a pharynx protector according to a second embodiment of the present invention; and

FIGS. 3a to 3c illustrate a pharynx protector according to a third embodiment of the present invention.

### DETAILED DESCRIPTION

The following description focuses on embodiments of the present invention applicable to a pharynx protector for receiving a puncture needle during puncture of the tracheoesophageal wall. The needle is used to create the puncture and to facilitate the subsequent introduction of a guide wire, which is inserted through the puncture needle, for example to insert a voice prosthesis in the tracheoesophageal wall by the aid of a dilator. A guidewire-dilator-prosthesis assembly is then pulled through the puncture site, dilating the puncture to facilitate the subsequent prosthesis placement.

For this purpose a pharynx protector for protecting the oesophagus and/or pharynx during performance of a laryngectomy is provided. The pharynx protector has a proximal end and a distal end, said distal end being adapted to be inserted into the oesophagus of a patient. The pharynx protector comprises an elongated tubular part and a distally located receiving portion, for receiving a puncture needle during laryngectomy. The tubular part is flexible, and the receiving portion comprises an opening, communicating with the lumen of the tubular part, such that a guide wire may be inserted through the opening and further passed proximally out of the pharynx protector. The surface of the inner wall of the receiving portion, having a normal passing through said opening, is of a rigid material.

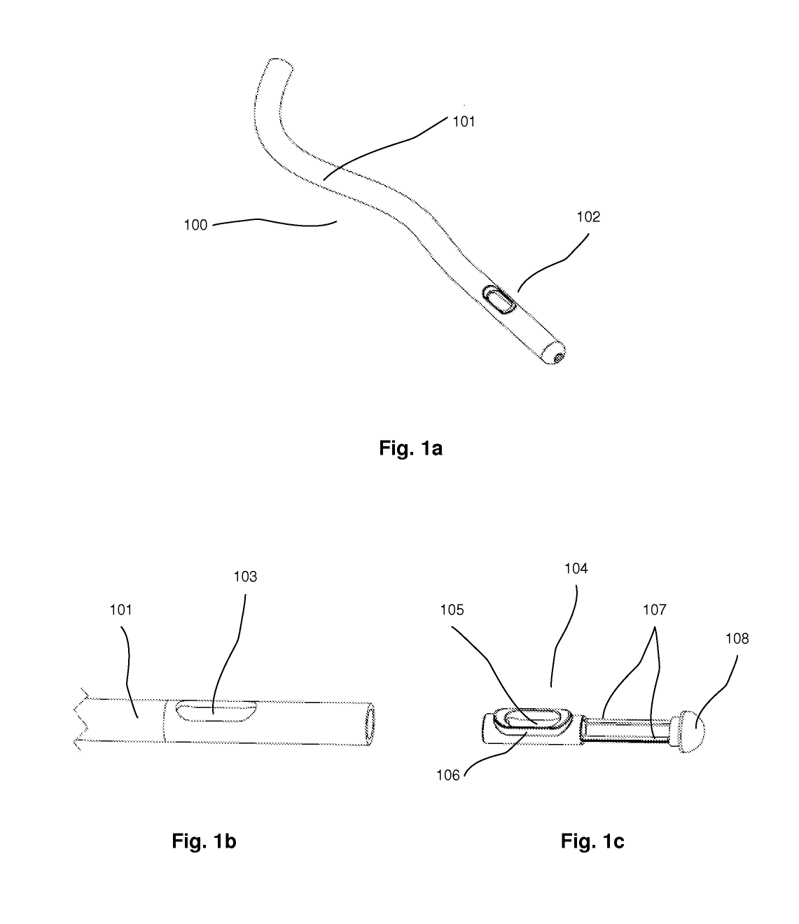

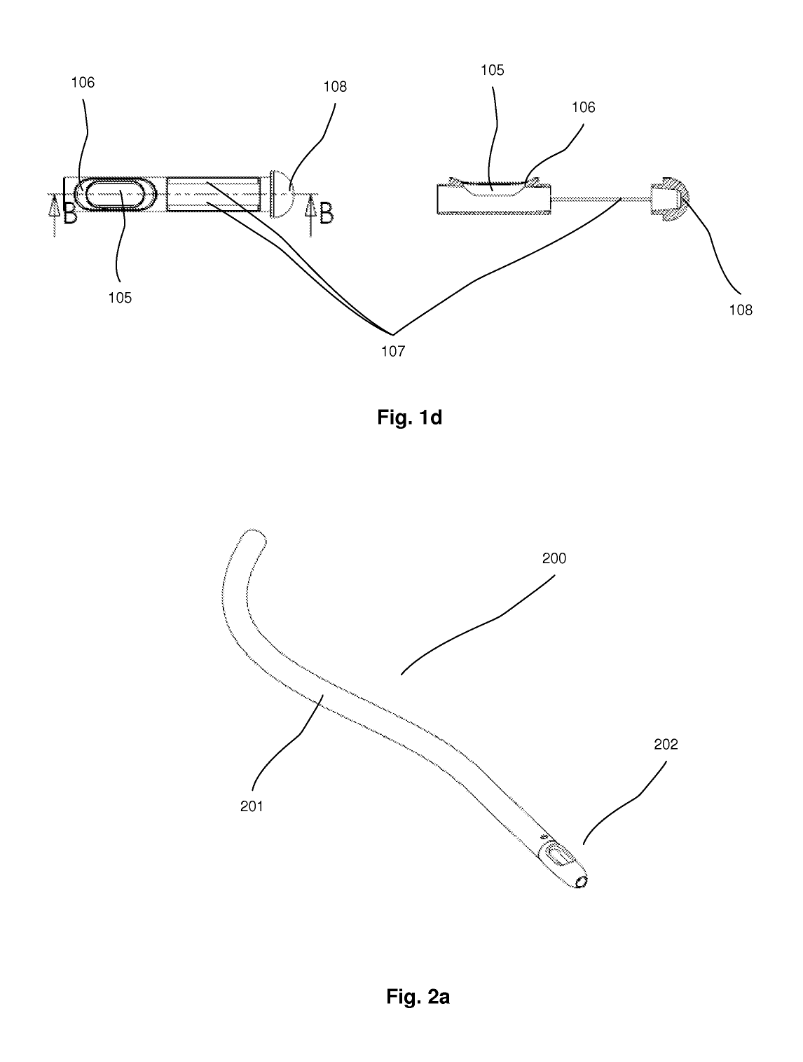

During secondary puncture, i.e. when a total laryngectomy has been performed without a voice prosthesis being placed at the time of surgery, a pharynx protector 100, comprising a flexible tube 101, according to FIG. 1a, is inserted into the oesophagus of the patient. The tube 101 is flexible to facilitate insertion of the tube 101 in the oesophagus of the patient. To facilitate use and decrease discomfort to the patient the tube 101 should have a flexability sufficient to exit the mouth of the patient while still extending down into oesophagus, without too strong bending having to be resisted by the body of the patient.

Close to a distal end of the flexible tube 101, the pharynx protector 100 is provided with a receiving portion 102, for receiving the puncture needle after said puncture needle has penetrated the tracheoesophageal wall.

The receiving portion 102 comprises an opening 103 in the tubular wall of the flexible tube 101, in accordance with FIG. 1b. The distal end of the flexible tube 101 is the end of the pharynx protector that is inserted through the mouth of the patient.

The receiving portion further comprises a rigid tubular element 104, inserted into the lumen of the flexible tube 101, in accordance with FIG. 1c. FIG. 1d illustrates a cross section along a plane B. The rigidity of the tubular element 104 should be such that it a puncture needle cannot penetrate through the wall of the tubular element 104 during practical circumstances. Thus, the puncture needle cannot penetrate the back wall of the tubular element 104, whereby the back wall of the oesophagus is protected.

The tubular element 104 has an opening 105 in the tubular wall thereof. The opening 105 corresponds in shape and size to the opening 103 in the tube 101. The opening 105 may have a circumferential rim 106 on the outside surface of the tubular element 104. This rim 106 may be adapted to securingly fit within the opening 103, such that the tubular element 104 may be snap fitted to the tube 101 by insertion through the distal end of the tube 101. In this way, the tubular element 104 may be secured to the tube 101 and thereby the pharynx protector 100.

The lumen of the tubular element 104, when inserted in the tube 101, communicates with the lumen of the tube 101, such that a guide wire may be inserted through the openings 103 and 105 and further passed proximally out of the pharynx protector and thereby out of the mouth of the patient.

The openings 103 and 105 may have an oval shape, which will increase the target area for the puncture needle penetrating the tracheoesophagal wall.

In the distal end of the tubular element 104, when arranged in the tube 101, the tubular element 105 may be provided with extension elements, in form of bars 107, extending coaxially with the central axis of the tubular element 104 in a distal direction. The bars 107 then corresponds to the length between the distal end of the opening 103 in the tube 101 and the distal end of the tube 101. In the distal end of the bars 107 a tip element 108 may be provided, such that the distal opening of the tube 101 is closed when the tubular element 104 is inserted into the tube 101.

The outer surface of the tip element 108 may be rounded, such as semi-spherical or dome shaped. Such shape will facilitate insertion of the pharynx protector through the mouth of the patient, and also facilitate the travel through the oesophagus of the patient to the target site. The tip element 108 may also be provided with an axial opening, having a direction axial to the central axis of the tube 101 and the tubular element 104. This axial opening may be used for inspection with a flexible endoscope. The axial opening may also be used if a guide wire is used for insertion of the pharynx protector, making it easier to introduce the pharynx protector in a difficult throat with strictures and other anatomical defects.

The bars 107 may be of such dimensions as to allow a certain flexibility, even though manufactured in the same rigid material as the rest of the tubular element 104. When the tubular element 104 is provided with bars 107, the distal part of the pharynx protector will be more flexible compared to the one lacking such extension elements, leading to decreased inconvenience during insertion of the pharynx protector. Also, the tubular element 104 and tip element 108 may be manufactured as one piece, making the pharynx protector cheaper, compared to having a separate tip element 108.

It is however possible to omit the extension elements 107, whereby the tip element 108 is arranged at the end of the tubular element 104. Thus, the extension elements 107 have been replaced by an extended part of the tubular element 104. In this way the tubular element 104 will form a barrel with an opening 105 in the tubular part of the barrel. Of course, the rest of the features relating to FIGS. 1a to 1d are equally applicable to the barrel, without departing from the scope of the invention.

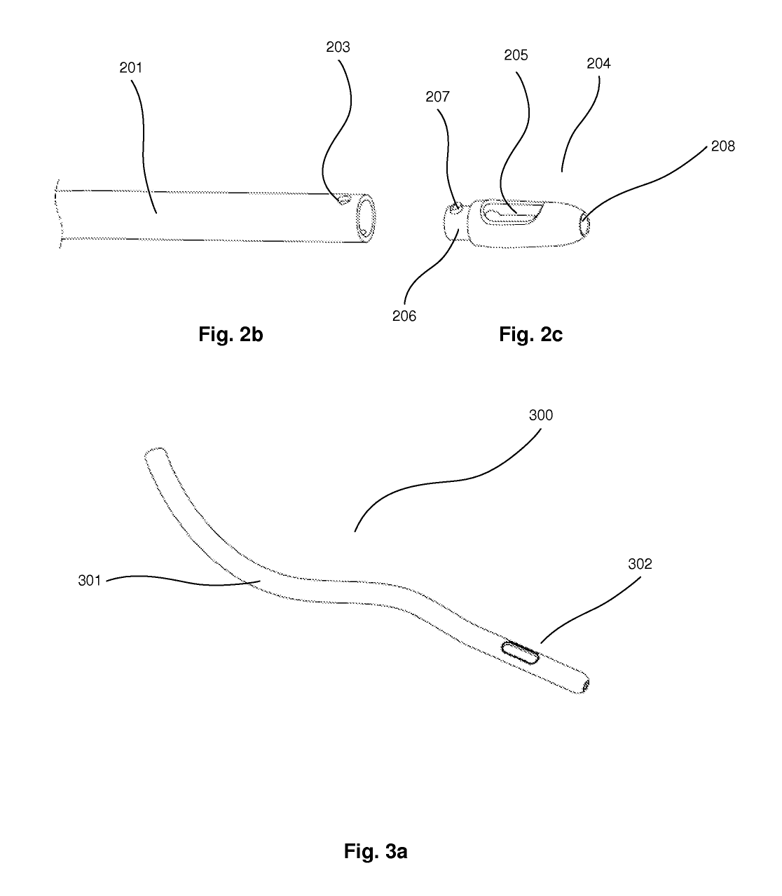

In another embodiment, according to FIGS. 2a to 2c, a pharynx protector 200 comprises a flexible tube 201, provided with a receiving portion 202 at the distal end thereof. The receiving portion 202 is a portion for receiving a puncture needle after said puncture needle has penetrated the tracheoesophageal wall.

The tube 201 is provided with two or more openings 203, in accordance with FIG. 2b, in the distal end thereof. The openings 203 constitute an attaching means, for securingly attaching a rigid tubular element 204 to the tube 201.

receiving portion 202 comprises an opening 205 in the tubular wall of the tubular element 204, in accordance with FIG. 2c. The opening 205 is adapted in shape and size for reception of a puncture needle after the puncture needle has penetrated/punctured the tracheoesophageal wall, in conformity with the openings 103 and 105 in respect of the embodiment disclosed in FIGS. 1a to 1d.

The opening 205 may have an oval shape, which will increase the target area for the puncture needle penetrating the tracheoesophagal wall.

The tubular element 204 is provided with a proximal part 206 corresponding in shape and size to the lumen of the tube 201. The proximal part may thus be inserted into the lumen of the tube 201. When inserted into the lumen of the tube 201, the lumen of the tubular element 204, communicates with the lumen of the tube 201, such that a guide wire may be inserted through the opening 205 and further passed proximally out of the pharynx protector and thereby out of the mouth of the patient.

On the outside of the proximal part 206 protrusions 207 extends outwardly from the proximal part 206. The protrusions 207 correspond in size and shape to the through openings 203 in the tube 201. These protrusions 207 may be adapted to securingly fit within the openings 203, such that the tubular element 204 may be snap fitted to the tube 201 by insertion of the proximal part 206 through the distal end of the tube 201. In this way, the tubular element 204 may be secured to the tube 201 and thereby the pharynx protector 200.

In the distal end of the tubular element 204 a tip element 208 may be provided, such that the distal opening of the tubular element 204 is closed.

The outer surface of the tip element 208 may be rounded, such as semi-spherical or dome shaped. Such shape will facilitate insertion of the pharynx protector through the mouth of the patient, and also facilitate the travel through the oesophagus of the patient to the target site. The tip element 208 may also be provided with an axial opening, having a direction axial to the central axis of the tube 201 and the tubular element 204. This axial opening may be used for inspection with a flexible endoscope. The axial opening may also be used if a guide wire or catheter is used for insertion of the pharynx protector, making it easier to introduce the pharynx protector in a difficult throat with strictures and other anatomical defects.

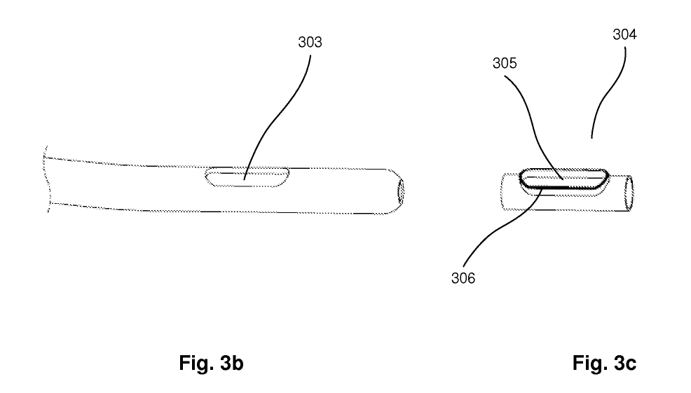

In another embodiment, according to FIGS. 3a to 3c, a pharynx protector 300 comprises a flexible tube 301, provided with a receiving portion 302 at the distal end thereof. The receiving portion 302 is a portion for receiving a puncture needle after said puncture needle has penetrated the tracheoesophageal wall. To facilitate use and decrease discomfort to the patient the tube 301 should have a flexability sufficient to exit the mouth of the patient while still extending down into oesophagus, without too strong bending having to be resisted by the body of the patient

Close to a distal end of the flexible tube 301, the pharynx protector 300 is provided with a receiving portion 302, for receiving the puncture needle after said puncture needle has penetrated the tracheoesophageal wall.

The receiving portion 302 comprises an opening 303 in the tubular wall of the flexible tube 301, in accordance with FIG. 3b. The distal end of the flexible tube 301 is the end of the pharynx protector that is inserted through the mouth of the patient.

The receiving portion further comprises a rigid tubular element 304, inserted into the lumen of the flexible tube 301, in accordance with FIGS. 3a and 3c. The rigidity of the tubular element 304 should be such that it a puncture needle cannot penetrate through the wall of the tubular element 304 during practical circumstances. Thus, the puncture needle cannot penetrate the back wall of the tubular element 304, whereby the back wall of the oesophagus is protected.

The tubular element 304 has an opening 305 in the tubular wall thereof. The opening 305 corresponds in shape and size to the opening 303 in the tube 301. The opening 305 may have a circumferential rim 306 on the outside surface of the tubular element 304. This rim 306 may be adapted to securingly fit within the opening 303, such that the tubular element 304 may be snap fitted to the tube 301 by insertion through the distal end of the tube 301. In this way, the tubular element 304 may be secured to the tube 301 and thereby the pharynx protector 300.

The lumen of the tubular element 304, when inserted in the tube 301, communicates with the lumen of the tube 301, such that a guide wire may be inserted through the openings 303 and 305 and further passed proximally out of the pharynx protector and thereby out of the mouth of the patient.

The openings 303 and 305 may have an oval shape, which will increase the target area for the puncture needle penetrating the tracheoesophagal wall.

A tubular rounded tip may be formed by heat forming the tube 301, whereby the tube shrinks, and forms a rounded tip shape, when further enclosing the tubular element 304. Also, this may further improve the securement of the tubular element 304 to the tube 301 and thereby the pharynx protector 300. The outer surface of the rounded tip may thus be rounded, such as semi-spherical or dome shaped. Such shape will facilitate insertion of the pharynx protector through the mouth of the patient, and also facilitate the travel through the oesophagus of the patient to the target site. The rounded tip may also be provided with an axial opening, having a direction axial to the central axis of the tube 301 and the tubular element 304. This axial opening may be used for inspection with a flexible endoscope. The axial opening may also be used if a guide wire is used for insertion of the pharynx protector, making it easier to introduce the pharynx protector in a difficult throat with strictures and other anatomical defects.

The tube 101, 201, 301 is made of a flexible material for a non-traumatic insertion into the oesophagus, through the pharynx. A suitable material for the tube 101, 201, 301 is a thermoplastic elastomer. A suitable thermoplastic elastomer may be selected from the group comprising styrene block copolymers (SBS; SEBS), thermoplastic polyurethanes, thermoplastic copolyesters, thermoplastic copolyamides, elastomeric polyolefines, thermoplastic polyolefine elastomer blends, vulcanized thermoplastic polyolefine elastomers, plasticized polyvinyl chloride, blends between elastomers, such as SBS, SEBS, etc., and plastics, such as polyethylene, polypropylene, polystyrene, ethylene vinyl acetate, etc., or any combination of these. MEDIPRENE™ is an example of a suitable material, being a blend of SEBS, paraffinic oil, and polypropylene.

The tubular element 104, 204, 304 may be manufactured of a rigid material, such as rigid plastic material having a suitable hardness for the intended purpose, i.e. to ensure that the puncture needle does not penetrate the back wall of the head portion. A suitable material may for example be polyoxymethylene, polypropylene, a polyamide, and a polycarbonate.

The diameter of the axial opening in the tip 108, 208 or the rounded tip in FIGS. 3a to 3c may suitably be selected to be in the interval of 2 to 8 mm, such as 4 to 6 mm.

The diameter of the flexible tube 101, 201, 301 may suitably be selected to be in the interval of 10 to 20 mm, such as 10 to 15 mm.

The length of the pharynx protector 100, 200, 300 may suitably be selected to be in the interval of 150 to 500 mm, such as 200 to 400 mm.

The diagonal of the openings 103, 105, 203, 205, 303, 305 may suitably be selected to be in the interval of 10 to 20 mm.

Of course, other additive attachment means, such as gluing, may be combined with the configuration of openings, rims, and protrusions, respectively, with regard the different embodiments disclosed herein.

The pharynx protector 100, 200, 300 is inserted into oesophagus of the patient, for example by swallowing. The opening 105, 205, 305 is positioned at the intended position of puncture. This can be assured by inserting a finger through the tracheostoma of the patient and palpating the outside of the oesophagus to thereby feel when the opening has the correct orientation. This can be achieved, since the oesophagus has flexible walls. Alternatively, a fiber endoscope may be inserted through the pharynx protector 100, 200, 300, and illuminating the position of puncture with a light that penetrates the tracheal-oesophagal wall. After puncturing the intended position with a puncture needle, the guide wire is inserted through the puncture needle into the pharynx protector 100, 200, 300. Thereafter, the puncture needle may be extracted and the guide wire may be pushed until it exits the mouth of the patient, whereafter the pharynx protector 100, 200, 300 may be extracted from the oesophagus of the patient.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A pharynx protector comprising: a proximal end and a distal end, the distal end being adapted to be inserted into an oesophagus of a patient, an elongated tubular part and a distally located receiving portion for receiving a puncture needle during a laryngectomy, wherein said tubular part is substantially flexible, and said receiving portion; including a first opening, communicating with a lumen of said tubular part, such that a guide wire may be inserted through the first opening and further passed proximally out of said pharynx protector; and a surface of an inner wall being of a rigid material.

2. The pharynx protector according to claim 1, wherein the distal end is rounded.

3. The pharynx protector according to claim 1, wherein the distal end has an axial opening.

4. The pharynx protector according to claim 1, wherein said receiving portion includes a rigid tubular element positioned in the lumen of said tubular part, said tubular element including a first opening positioned underneath a second opening in said tubular part.

5. The pharynx protector according to claim 4, wherein the first and the second openings are corresponding in size and shape.

6. The pharynx protector according to claim 4, wherein said tubular element includes a rim on an outside surface of said tubular element encircling the first opening and fitting within the second opening of said tubular part.

7. The pharynx protector according to claim 4, wherein said tubular element includes bars extending distally and coaxially with a central axis of said tubular element and a tip element in the distal end thereof.

8. The pharynx protector according to claim 1, wherein the distal end has been heat formed, and further wherein the distal end of said tubular part is rounded.

9. The pharynx protector according to claim 1, wherein said receiving portion includes a rigid tubular element including the first opening, said tubular element being positioned distally of said tubular part.

10. The pharynx protector according to claim 9, wherein a proximal part of said tubular element is inserted into the lumen of said tubular part, and protrusions extending outwardly from said proximal part are fitted within openings in the distal end of said tubular part.

11. The pharynx protector according to claim 9, wherein said tubular element is provided with a tip element in the distal end thereof.

12. The pharynx protector according to claim 1, wherein the first opening has an oval shape.

13. The pharynx protector according to claim 1, wherein said tubular part is made at least in part of a thermoplastic elastomer.

14. The pharynx protector according to claim 1, wherein said tubular part is made of at least one of styrene block copolymers, thermoplastic polyurethanes, thermoplastic copolyesters, thermoplastic copolyamides, elastomeric polyolefines, thermoplastic polyolefine elastomer blends, yulcanized thermoplastic polyolefine elastomers, plasticized polyvinyl chloride, blends between elastomers, and plastics.

15. The pharynx protector according to claim 1, wherein said tubular part is made of a blend of at least one of SEBS, paraffinic oil, and polypropylene.

16. The pharynx protector according to claim 1, wherein the rigid material is at least one of polyoxymethylene, polypropylene, polyamides, a polyamide, and a polycarbonate.

17. The pharynx protector according to claim 1, wherein said tubular part has a diameter between 10 and 15 mm.

18. The pharynx protector according to claim 1, wherein the length of said pharynx protector is between 200 and 400 mm.

19. The pharynx protector according to claim 1, wherein the first opening has a diagonal between 10 and 20 mm.

20. The pharynx protector according to claim 3, wherein the axial opening is at least 4 mm.
